(19) United States
(12) Patent Application Publication
(10) Pub. No.: US 2024/0269513 A1
(43) Pub. Date: Aug. 15, 2024

(54) SYSTEM AND METHOD FOR TRACKING AND RECOMMENDING BREATHING EXERCISES USING WEARABLE DEVICES

(71) Applicant: Samsung Electronics Co., Ltd., Suwon-si (KR)

(72) Inventors: Md Mahbubur Rahman, San Jose, CA (US); Tousif Ahmed, San Jose, CA (US); Nafiul Rashid, Irvine, CA (US); Jilong Kuang, San Jose, CA (US); Jun Gao, Menlo Park, CA (US)

(21) Appl. No.: 18/358,769

(22) Filed: Jul. 25, 2023

Related U.S. Application Data
(60) Provisional application No. 63/445,638, filed on Feb. 14, 2023.

(51) Int. Cl. A63B 24/00; A63B 23/18; A63B 71/06; G16H 50/20

(52) U.S. Cl. CPC A63B 24/0075; A63B 23/18; A63B 71/0622; G16H 50/20; A63B 2024/0068; A63B 2220/40; A63B 2220/803; A63B 2220/836; A63B 2230/42

(57) ABSTRACT

A method includes collecting motion data of a user using a head-worn device while the user is performing a breathing exercise. The method also includes, for a window of the motion data, generating breathing depth features based on the motion data. The method further includes determining, using a first machine learning model that receives the breathing depth features as inputs, whether the motion data corresponds to a non-breathing motion. In addition, the method includes, responsive to determining that the motion data corresponds to the non-breathing motion, presenting a first notification to the user to adjust head motion.

### CROSS-REFERENCE TO RELATED APPLICATION AND PRIORITY CLAIM

This application claims priority under 35 U.S.C. § 119(e) to U.S. Provisional Patent Application No. 63/445,638 filed on Feb. 14, 2023, which is hereby incorporated by reference in its entirety.

### TECHNICAL FIELD

This disclosure relates generally to electronic health monitoring systems and processes. More specifically, this disclosure relates to a system and method for tracking and recommending breathing exercises using wearable devices.

### BACKGROUND

Numerous mobile applications (“apps”) are available that claim to help a user recover from stress. These applications generally facilitate user-generic recovery activities, such as breathing exercises, yoga, calming pictures and videos, and the like. These applications typically do not monitor actual meditative activities (such as mindful breathing) and do not provide user feedback as to whether such meditative exercises are performed properly.

### SUMMARY

This disclosure provides a system and method for tracking and recommending breathing exercises using wearable devices.

In a first embodiment, a method includes collecting motion data of a user using a head-worn device while the user is performing a breathing exercise. The method also includes, for a window of the motion data, generating breathing depth features based on the motion data. The method further includes determining, using a first machine learning model that receives the breathing depth features as inputs, whether the motion data corresponds to a non-breathing motion. In addition, the method includes, responsive to determining that the motion data corresponds to the non-breathing motion, presenting a first notification to the user to adjust head motion.

In a second embodiment, an electronic device includes at least one processing device configured to collect motion data of a user using a head-worn device while the user is performing a breathing exercise. The at least one processing device is also configured, for a window of the motion data, to generate breathing depth features based on the motion data. The at least one processing device is further configured to determine, using a first machine learning model that receives the breathing depth features as inputs, whether the motion data corresponds to a non-breathing motion. In addition, the at least one processing device is configured, responsive to determining that the motion data corresponds to the non-breathing motion, to present a first notification to the user to adjust head motion.

In a third embodiment, a non-transitory machine-readable medium contains instructions that when executed cause at least one processor of an electronic device to collect motion data of a user using a head-worn device while the user is performing a breathing exercise. The medium also contains instructions that when executed cause the at least one processor, for a window of the motion data, to generate breathing depth features based on the motion data. The medium further contains instructions that when executed cause the at least one processor to determine, using a first machine learning model that receives the breathing depth features as inputs, whether the motion data corresponds to a non-breathing motion. In addition, the medium contains instructions that when executed cause the at least one processor, responsive to determining that the motion data corresponds to the non-breathing motion, to present a first notification to the user to adjust head motion.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate.” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B.” “at least one of A and/or B,” or “one or more of A and/or B” may include all possible combinations of A and B. For example, “A or B.” “at least one of A and B.” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an.” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch). Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a drier, an air cleaner, a set-top box, a home automation control panel, a security control panel, a TV box (such as SAMSUNG HOMESYNC, APPLETV, or GOOGLE TV), a smart speaker or speaker with an integrated digital assistant (such as SAMSUNG GALAXY HOME, APPLE HOMEPOD, or AMAZON ECHO), a gaming console (such as an XBOX, PLAYSTATION, or NINTENDO), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. According to some embodiments of this disclosure, the electronic device may be a flexible electronic device. The electronic device disclosed here is not limited to the above-listed devices and may include new electronic devices depending on the development of technology.

In the following description, electronic devices are described with reference to the accompanying drawings, according to various embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle. Use of any other term, including without limitation “mechanism,” “module,” “device.” “unit,” “component,” “element,” “member,” “apparatus,” “machine.” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description taken in conjunction with the accompanying drawings, in which like reference numerals represent like parts:

FIG. 1 illustrates an example network configuration including an electronic device according to this disclosure;

FIG. 2 illustrates an example system for tracking and recommending breathing exercises using wearable devices according to this disclosure;

FIGS. 3A and 3B illustrate example processes for real-time on-bud phase tracking according to this disclosure;

FIG. 4 illustrates an example chart showing raw accelerometer data during a user's breathing exercise session according to this disclosure;

FIG. 5 illustrates an example process for real-time breathing depth determination according to this disclosure;

FIGS. 6A through 6D illustrate example screen images of a user interface that can be used during a guided breathing exercise according to this disclosure;

FIG. 7 illustrates an example process for breath-hold detection according to this disclosure;

FIG. 8 illustrates an example process for determining passive breathing conditions for breathing exercise recommendation according to this disclosure;

FIG. 9 illustrates an example process for passive breath tracking according to this disclosure;

FIG. 10 illustrates an example process for passive breathing depth estimation according to this disclosure;

FIG. 11 illustrates an example process for breath tracking using inertial measurement unit (IMU) and audio data according to this disclosure;

FIG. 12 illustrates an example process for breath tracking using audio data according to this disclosure; and

FIG. 13 illustrates an example method for tracking and recommending breathing exercises using at least one wearable device according to this disclosure.

### DETAILED DESCRIPTION

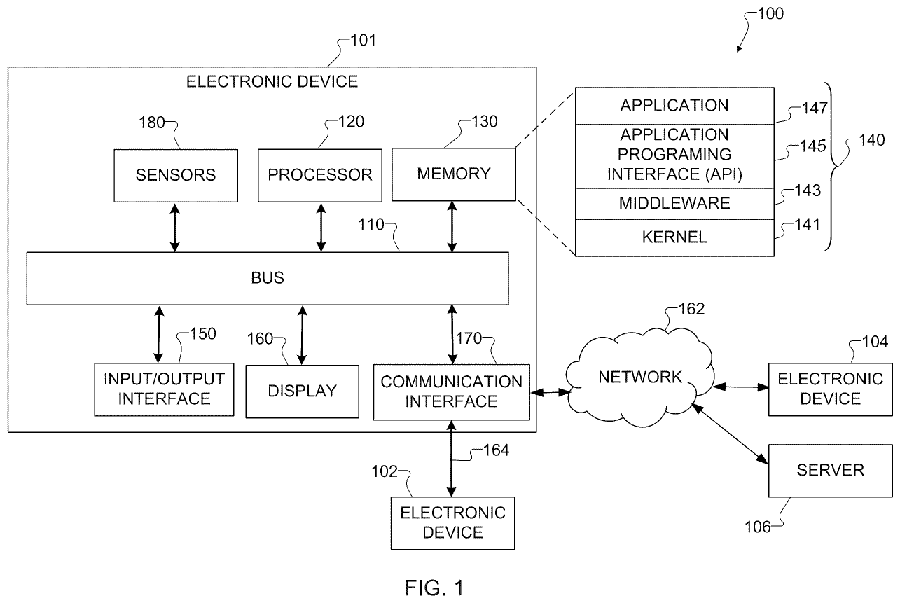

FIGS. 1 through 13, discussed below, and the various embodiments of this disclosure are described with reference to the accompanying drawings. However, it should be appreciated that this disclosure is not limited to these embodiments and all changes and/or equivalents or replacements thereto also belong to the scope of this disclosure.

As discussed above, numerous mobile applications (“apps”) are available that claim to help a user recover from stress. These applications generally facilitate user-generic recovery activities, such as breathing exercises, yoga, calming pictures and videos, and the like. These applications typically do not monitor actual meditative activities (such as mindful breathing) and do not provide user feedback as to whether such meditative exercises are performed properly.

One example of a meditative activity is mindful breathing Mindful breathing helps to connect the mind and body and put someone in a proper state through the use of controlled breathing cycles. A typical breathing cycle used in mindful breathing includes an inhalation, holding the breath, an exhalation, and sometimes holding the breath after exhaling. Different mindful breathing exercises can be performed for varying intents. For example, equal breaths (“Sama Vittri”), coherence breathing, and box-breathing (four-second inhale, four-second hold, four-second exhale, and four-second hold) are supposed to improve relaxation. “Breath of fire” breathing is used to increase calmness, “ocean breath” (also called “ujjayi”) is used to empower focus, and “4-7-8 breath” (four-second inhale, seven-second hold, and eight-second exhale) is used to reduce anxiety.

Despite their intended benefits, some mindful breathing exercises can fail and actually exacerbate stress if not performed properly. Studies have shown that some people with no prior exposure to breathing exercises can perform mindful breathing exercises incorrectly and end up with higher levels of stress. This is called the “meditation paradox.” Moreover, traditionally relaxing breathing exercises are self-initiated and self-tracked, which can distract the user from the meditative exercises. For example, a user having to count his or her breaths while performing the breathing exercises can easily lose count or become distracted. Hence, one of the biggest challenges in mindful breathing is for the user to maintain focus on the breathing. Other issues with mindful breathing exercises include a lack of user understanding of how meditation works and physical discomfort (such as chest tightness). In addition, some mindful breathing exercises ideally need an exercise therapist to select a suitable exercise and adjust the exercise over time, which is typically very expensive and not available everywhere.

This disclosure provides various techniques for tracking and recommending breathing exercises using wearable devices. As described in more detail below, the disclosed systems and methods estimate a user's breathing depth for passive and meditative breathing exercises and determine objective breathing performance by combining multiple breathing biomarkers from breathing exercises. The disclosed systems and methods also capture the breathing motion, determine whether breathing is shallow or deep, and trigger breathing exercises for meditation. In addition, the disclosed systems and methods can track the breathing exercises performed by the user and passively determine the user's performance to follow a particular exercise. This can help to overcome at least some of the issues noted above. Note that while some of the embodiments discussed below are described in the context of use in consumer electronic devices (such as smart earbuds or smartphones), this is merely one example, and it will be understood that the principles of this disclosure may be implemented in any number of other suitable contexts and may use any suitable devices.

FIG. 1 illustrates an example network configuration 100 including an electronic device according to this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more processing devices, such as one or more microprocessors, microcontrollers, digital signal processors (DSPs), application specific integrated circuits (ASICs), or field programmable gate arrays (FPGAs). In some embodiments, the processor 120 includes one or more of a central processing unit (CPU), an application processor (AP), a communication processor (CP), or a graphics processor unit (GPU). The processor 120 is able to perform control on at least one of the other components of the electronic device 101 and/or perform an operation or data processing relating to communication or other functions. As described in more detail below, the processor 120 may perform one or more operations for tracking and recommending breathing exercises using one or more wearable devices.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. The application 147 may support one or more functions for tracking and recommending breathing exercises using one or more wearable devices as discussed below. These functions can be performed by a single application or by multiple applications that each carry out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143. For example, the API 145 includes at least one interface or function (such as a command) for filing control, window control, image processing, or text control.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals.

The wireless communication is able to use at least one of, for example, WiFi, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a communication protocol. The wired connection can include, for example, at least one of a universal serial bus (USB), high definition multimedia interface (HDMI), recommended standard 232 (RS-232), or plain old telephone service (POTS). The network 162 or 164 includes at least one communication network, such as a computer network (like a local area network (LAN) or wide area network (WAN)), Internet, or a telephone network.

The electronic device 101 further includes one or more sensors 180 that can meter a physical quantity or detect an activation state of the electronic device 101 and convert metered or detected information into an electrical signal. For example, one or more sensors 180 can include one or more cameras or other imaging sensors for capturing images of scenes. The sensor(s) 180 can also include one or more buttons for touch input, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

In some embodiments, the electronic device 101 can be a wearable device or an electronic device-mountable wearable device (such as an HMD). For example, the electronic device 101 may represent an AR wearable device, such as a headset with a display panel or smart eyeglasses. In other embodiments, the first external electronic device 102 or the second external electronic device 104 can be a wearable device or an electronic device-mountable wearable device (such as an HMD). In those other embodiments, when the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving a separate network.

The first and second external electronic devices 102 and 104 and the server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith. The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162 or 164, the electronic device 101 may be independently operated without a separate communication function according to some embodiments of this disclosure.

The server 106 can include the same or similar components 110-180 as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101. As described in more detail below, the server 106 may perform one or more operations to support techniques for tracking and recommending breathing exercises using one or more wearable devices.

Although FIG. 1 illustrates one example of a network configuration 100 including an electronic device 101, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

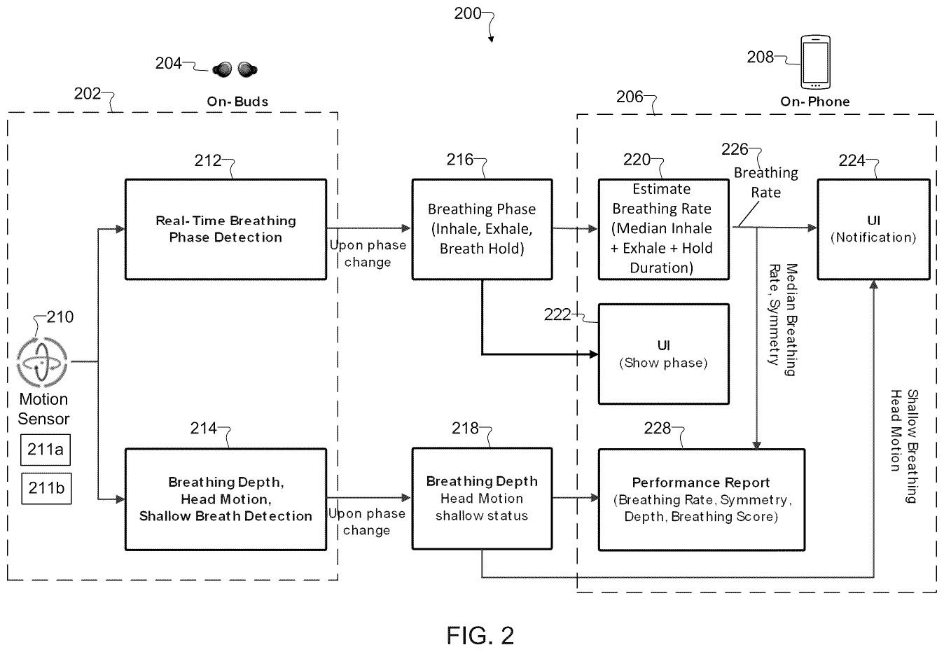

FIG. 2 illustrates an example system 200 for tracking and recommending guided breathing exercises using wearable devices according to this disclosure. For ease of explanation, the system 200 is described as being implemented using one or more components of the network configuration 100 of FIG. 1 described above, such as the electronic device 101. However, this is merely one example, and the system 200 could be implemented using any other suitable device(s) and in any other suitable system(s).

As shown in FIG. 2, the system 200 includes an on-bud module 202 that is executed on one or more earbuds 204 worn by a user and an on-phone module 206 that is executed on a smartphone 208 associated with the user. The earbuds 204 and the smartphone 208 are communicatively coupled to each other via a wired or wireless connection and can share data using a suitable communication protocol, such as BLUETOOTH or any other suitable protocol. Each of the earbuds 204 and the smartphone 208 can represent (or be represented by) the electronic device 101 of FIG. 1.

The one or more earbuds 204 include a multi-axis (such as a six-axis) motion sensor 210 that may include an accelerometer 211a and a gyroscope 211b. In some cases, the accelerometer 211a may be a three-axis accelerometer, and the gyroscope 211b may be a three-axis gyroscope (although other configurations are possible). The motion sensor 210 senses breathing movement of the user and converts breathing movement information into motion data that is input to a breathing phase detection module 212 and a breathing depth detection module 214, which are part of the on-bud module 202. As described in greater detail below, the breathing phase detection module 212 uses the motion data to detect the user's breathing phase 216 and related information, and the breathing depth detection module 214 uses the motion data to detect the user's breathing depth 218 and related information. The breathing phase 216, breathing depth 218, and related information are transmitted to the on-phone module 206 (such as by using BLUETOOTH or another suitable communication protocol) for post-processing and for use in determining the user's breathing rate 226 and guiding the user's breathing pattern.

### Breathing Phase Detection

The breathing phase detection module 212 operates in real-time on one or more of the earbuds 204 to perform phase tracking. Phase tracking allows the breathing phase detection module 212 to detect the breathing phase of the user and detect a phase change (if any). The breathing phase information, including any detected phase change, and a corresponding timestamp may be transmitted to the on-phone module 206.

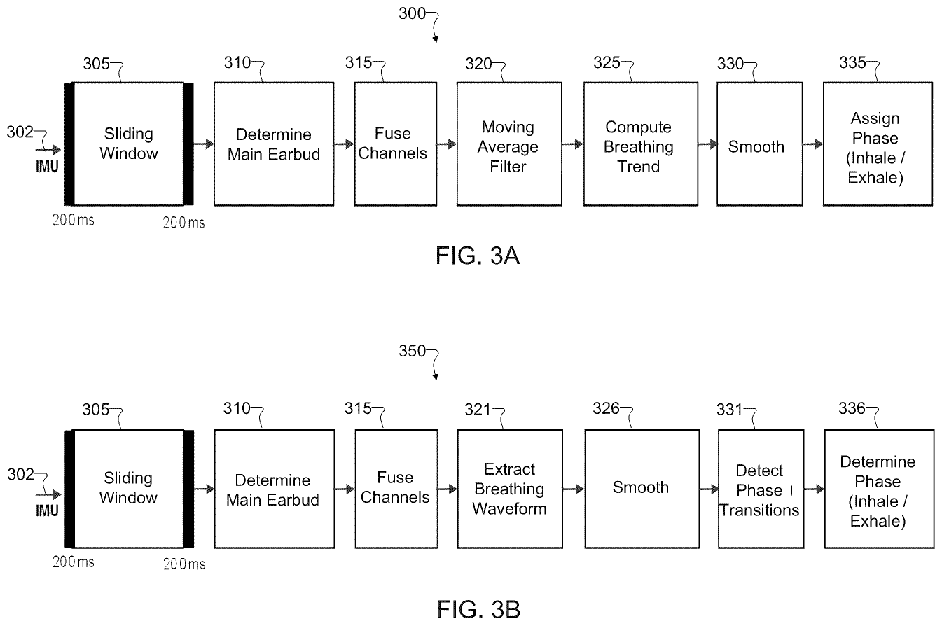

FIGS. 3A and 3B illustrate example processes 300 and 350 for real-time on-bud phase tracking according to this disclosure. In particular, FIG. 3A illustrates a process 300 using accelerometer data from the accelerometer 211a, and FIG. 3B illustrates a process 350 using gyroscope data from the gyroscope 211b. The processes 300 and 350 can be performed by the breathing phase detection module 212 of FIG. 2.

As shown in FIG. 3A, the process 300 uses a sliding window 305 of motion data 302 from the accelerometer 211a. The motion data 302 can include, for example, a three-axis signal from the accelerometer 211a. In some embodiments, the sliding window 305 is a two-second sliding window with a step size of 200 ms, which allows determination of the breathing phase every 200 ms. At operation 310, the breathing phase detection module 212 determines a main earbud by selecting either the left earbud 204 or the right earbud 204. Because the left and right earbuds 204 have orientation differences, the on-bud module 202 can adapt the motion direction algorithm based on the determined main earbud. At operation 315, the breathing phase detection module 212 fuses the three channels of the three-axis accelerometer signal. For many common breathing exercise positions that involve sitting upright, the breathing depth may mostly closely correspond to the amplitude of the X axis.

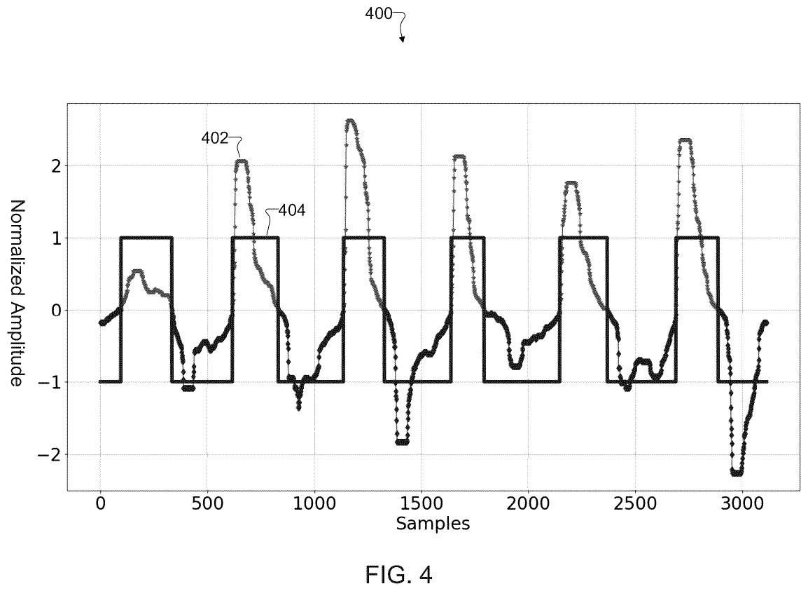

FIG. 4 illustrates an example chart 400 showing raw gyroscope data 402 during a user's breathing exercise session according to this disclosure. Example data 402 shown in the chart 400 was obtained from a left earbud 204 while a user was sitting upright. In this example, the user was performing a symmetrical breathing exercise with a six-second inhale and six-second exhale. In some embodiments, the accelerometer data 402 can represent the fused data obtained after the operation 315.

At operation 320, the breathing phase detection module 212 applies a rolling moving average filter, such as one with a one-second window size, to smooth out the raw signal and better extract the breathing trend. At operation 325, the breathing phase detection module 212 takes the first difference of the signal to compute the breathing trend, which may be done using the following.

$${Slope}_{t} = {S}_{t + 1} - {S}_{t}$$

Here, Slopeₜ is the derived slope at time/from the derived breathing waveform signal S. Given this, an upward trend signal generates positive slopes (values above the zero line in the chart 400), and a downward trend generates negative slopes (values below the zero line). The breathing phase detection module 212 can transform all positive values as +1 and all negative values as −1, such as by using the following.

$${Phase}_{t} = \{ \begin{matrix}- 1 , & if {Slope}_{t} < 0 \\ + 1 , & if {Slope}_{t} > 0\end{matrix}$$

The result is a square waveform 404 in the chart 400. However, due to occasional non-breathing head motion by the user, false spikes can be observed. At operation 330, the breathing phase detection module 212 performs smoothing to reduce any false detections. At operation 335, the breathing phase detection module 212 assigns an inhalation label to all +1 values and assigns an exhalation label to all −1 values. This assignment results in the breathing phase 216. In some embodiments, the predicted inhalation and exhalation phases timestamps can be compared with annotated inhalation and exhalation timestamps to evaluate the algorithm performance (such as during a training exercise).

As shown in FIG. 3B, the process 350 uses a sliding window 305 of motion data 302 from the gyroscope 211b. The motion data 302 can include, for example, a three-axis signal from the gyroscope 211b. In some embodiments, gyroscope data can be more robust to motion artifacts than accelerometer data. At operation 310, the breathing phase detection module 212 determines a main earbud by selecting either the left earbud 204 or the right earbud 204. At operation 315, the breathing phase detection module 212 fuses the three channels of the three-axis gyroscope signal. In the process 350, the breathing phase detection module 212 in this example selects the Z-axis since it produces the best accuracy.

At operation 321, the breathing phase detection module 212 extracts the breathing waveform by applying a median filter and normalizing the signal using z-score normalization. The breathing phase detection module 212 also applies the median filter again to extract the underlying breathing waveform. The breathing phase detection module 212 further transforms the signal into a square waveform (also considered as the phase signal), such as by using the following.

$${Phase}_{t} = \{ \begin{matrix}- 1 , & if {S}_{t} < 0 \\ + 1 , & if {S}_{t} > 0\end{matrix}$$

At operation 326, the breathing phase detection module 212 performs smoothing on the phase signal to remove the spikes due to motion artifacts. At operation 331, the breathing phase detection module 212 detects the phase transitions, such as by using the following.

$${Transition}_{t} = {Phase}_{t + 1} - {Phase}_{t}$$

At operation 336, the breathing phase detection module 212 determines the breathing phase 216 by assigning a positive-to-negative value transition as an inhalation phase and a negative-to-positive value transition as an exhalation phase.

The processes 300 and 350 described above can determine the duration of each phase in each breathing cycle based on the timestamp of the detected breathing phases. In some embodiments, the breathing phase detection module 212 can consider the duration of the consecutive +1 s as the inhalation duration and the duration of the −1 s as the exhalation duration. The breathing phase detection module 212 can also calculate breathing symmetry as the ratio between inhalation and exhalation duration. In some embodiments, the breathing phase detection module 212 calculates the breathing symmetry for each breathing cycle and takes the median of all breathing symmetries calculated in a breathing exercise session to determine an overall breathing symmetry. Breathing symmetry can be useful or important to track since target breathing exercises can be symmetrical or asymmetrical. If the user targets asymmetrical breathing exercises (such as shorter inhale and longer exhale), the breathing symmetry will be lower than one. If the user targets symmetrical breathing exercises (inhale and exhale have the same duration), the breathing symmetry should be close to one.

### Breathing Depth Detection

Like the breathing phase detection module 212, the breathing depth detection module 214 operates in real-time on one or more of the earbuds 204. In some embodiments, the breathing depth detection module 214 can use a ten-second non-overlapping sliding window to calculate the breathing depth 218 and to detect head motion and/or shallow breathing. In general, breathing depth can be a useful or important metric to distinguish deeper breathing exercises from regular shallow breathing. Breathing depth is an indicator of how much air is inhaled into the lungs while someone is breathing. Inhaled air is proportional to lung expansion, and lung expansion is proportional to the breathing head motion. Since the algorithm described below is designed to capture the breathing head motion, the amplitude of the extracted breathing signal can be considered as breathing depth. The head motion, shallow breathing, and breathing depth 218 (and optional corresponding timestamp) may be transmitted to the on-phone module 206 at regular or other intervals, such as every ten seconds.

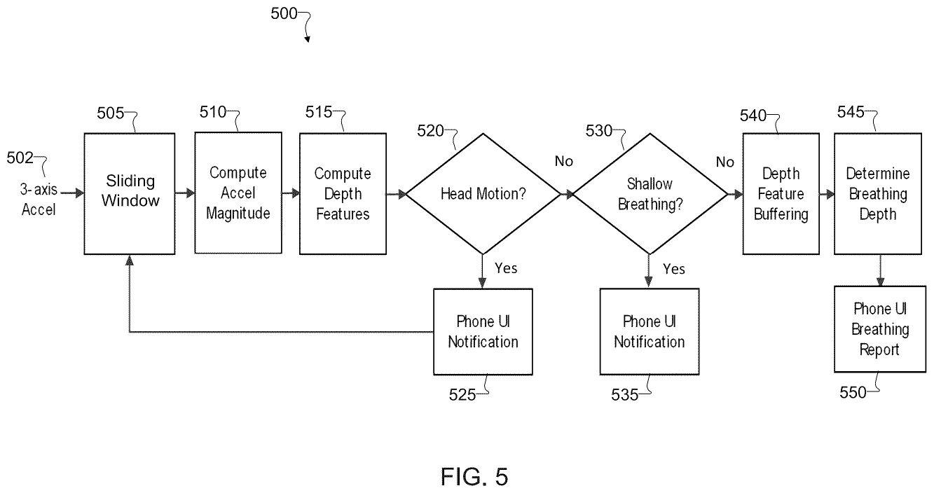

FIG. 5 illustrates an example process 500 for real-time breathing depth determination according to this disclosure. The process 500 can be performed by the breathing depth detection module 214 during breathing exercises by a user. The process 500 includes multiple functions, such as (1) shallow breathing detection for near real-time feedback on breathing depth and (2) overall normalized breathing depth determination in percentage at the end of the exercise. In some embodiments, portions of the end-of-the-exercise session normalized depth determination are performed by the on-phone module 206.

As shown in FIG. 5, the process 500 uses a sliding window 505 of motion data 502 from the accelerometer 211a. The motion data 502 can include, for example, a three-axis signal from the accelerometer 211a. In some embodiments, the sliding window 505 is a ten-second sliding window. The sliding window 505 can be longer than the sliding window 305 used in phase detection because the depth detection may involve a longer duration of data. In some embodiments, the maximum inhalation or exhalation duration in a particular breathing exercise is ten seconds to support slow-paced breathing (such as up to three breaths per minute). Therefore, it is expected that the ten-second sliding window 505 would capture at least one inhalation or one exhalation phase of a breathing cycle. Also, the breathing depth detection module 214 can detect head motion during the process 500, and the longer sliding window 505 can help to ensure that the head motion is consistent with the breathing and does not affect the breathing phase and depth detection.

At operation 510, the breathing depth detection module 214 computes the accelerometer magnitude range and the accelerometer Z-axis range from the sliding window 505 of motion data 502. The accelerometer magnitude range and the accelerometer Z-axis range are later used by the breathing depth detection module 214 for non-breathing head motion detection. In breathing, chest expansion and contraction may result in the user moving his or her shoulders and head. However, a person often moves his or her head for many other purposes, such as nodding or shaking the head from left to right. It can be useful or important to distinguish such non-breathing head motions when using earbud motion data to track breathing. The breathing depth detection module 214 collects data corresponding to the various non-breathing head movements to distinguish the non-breathing head motions from the breathing head motions.

At operation 515, the breathing depth detection module 214 extracts the breathing waveform by removing high-frequency components with a filter, such as a five-sample median filter. The breathing depth detection module 214 also computes multiple breathing depth features from the accelerometer data based on the sliding window 505 to create depth biofeedback, such as every ten seconds. Example breathing depth features may include minimum, maximum, range, percentile range (such as the difference between the 90^{th} percentile and the 10^{th} percentile), and variance on each accelerometer axis and the accelerometer magnitude (\sqrt{x²+y²+z²}). In some embodiments, the breathing depth detection module 214 can further compute multiple breathing depth features from the gyroscope data.

At operation 520, the breathing depth detection module 214 performs non-breathing head motion detection. It has been observed during experimentation that the accelerometer magnitude range and the accelerometer Z-axis range can be useful or important features computed on the sliding window 505 to distinguish non-breathing head motion from breathing motion. Thresholds can be determined or trained in advance for these two breathing depth features. The breathing depth detection module 214 can determine the presence of non-breathing head motion if either motion feature (accelerometer magnitude range and accelerometer Z-axis range) is above its corresponding threshold. In some embodiments, the breathing depth detection module 214 can employ a trained machine learning model that receives the breathing depth features and determines whether or not the motion data corresponds to non-breathing head motion. The machine learning model can be trained to be sensitive to non-breathing head motion since this can be a large confounding factor for the earbud motion sensor-based breathing exercise tracking. If the breathing depth detection module 214 detects non-breathing head motion, at operation 525, the breathing depth detection module 214 can send an instruction for user notification of non-breathing head motion on the smartphone 208 and discard the current window from further processing. In some embodiments, the notification on the smartphone 208 may include a user instruction to adjust or reduce head motion while performing the breathing exercise.

At operation 530, the breathing depth detection module 214 performs shallow breathing detection based on the breathing depth features obtained in operation 515. The breathing depth detection module 214 may determine each feature's importance in distinguishing shallow breathing from deep breathing and mindful breathing exercises. In some embodiments, the breathing depth detection module 214 inputs the breathing depth features into a classifier that has been trained to distinguish shallow breathing from deep breathing. The training of the classifier can include multiple training datasets that have labels of regular breathing, shallow breathing, and heavy and guided controlled breathing datasets. In some embodiments, the classifier includes a trained machine learning model. In particular embodiments, the classifier includes a random forest classifier using leave-one-subject-out cross-validation. However, any other suitable classifier can be used for shallow breathing detection. If the breathing depth detection module 214 detects shallow breathing, at operation 535, the breathing depth detection module 214 can send an instruction for user notification of shallow breathing on the smartphone 208. In some embodiments, the notification on the smartphone 208 may include a user instruction to breathe deeper while performing the breathing exercise.

At operation 540, the breathing depth detection module 214 buffers the breathing depth features in a data buffer. The breathing depth features are determined at a regular or other interval (such as every ten seconds), and the breathing depth features are buffered so that a time sequence of data is available for determining the breathing depth 218. At operation 545, the breathing depth detection module 214 or the on-phone module 206 determines the breathing depth 218. In embodiments where the on-phone module 206 determines the breathing depth 218, the on-phone module 206 receives the breathing depth features from the data buffer. For ease of explanation, the operation 545 will be described as being performed by the breathing depth detection module 214. In some embodiments, breathing depth detection module 214 calculates the breathing depth 218 as a percentage in order to promote user understanding. In particular embodiments, the breathing depth 218 is calculated based on the percentile range of the X-axis of the accelerometer amplitude (Accel Amplitude) since this feature may be a useful or important feature in distinguishing deep breathing from shallow breathing. As a particular example, the breathing depth detection module 214 may calculate an accelerometer amplitude max feature using the following.

$$Accel Amplitude Max = {Q}_{3} + 1.5 * ( {Q}_{3} - {Q}_{1} )$$

Here, Q₁ and Q₃ are the first and third quartile, respectively, of the inhalation amplitude derived from accelerometer data. Other embodiments can consider gyroscope data or other inertial sensor data instead of or in addition to the accelerometer data.

To make the depth information more insightful and actionable for the user, the breathing depth detection module 214 may normalize the depth information with respect to the same features extracted from a training dataset that includes breathing exercise results from multiple participants with varying age, gender, and ethnicity. Finally, the breathing depth detection module 214 can compute the breathing depth 218 as a percentage, such as by using the following.

$$Depth = 100 * \frac{Accel Amplitude Max}{λ} \%$$

Here, λ is the normalization factor trained from the dataset. The breathing depth 218 indicates how deep is the user's breathing in the current exercise compared to the maximum depth of breathing determined in the dataset. At operation 550, the breathing depth detection module 214 can provide the breathing depth 218 to the smartphone 208 for use in a breathing report as described in greater detail below.

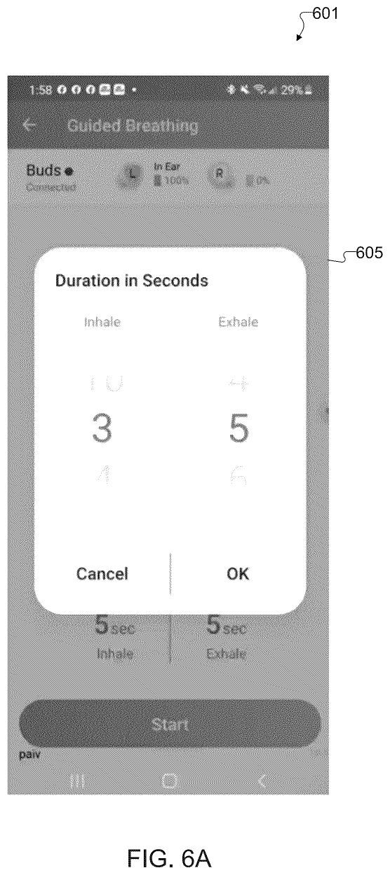

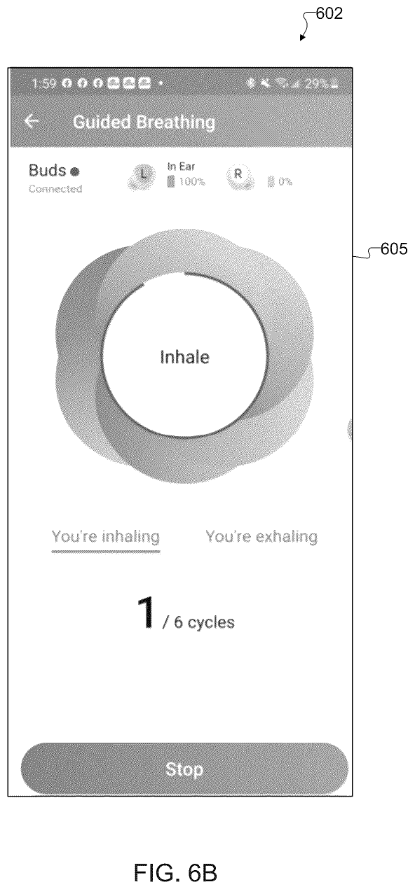

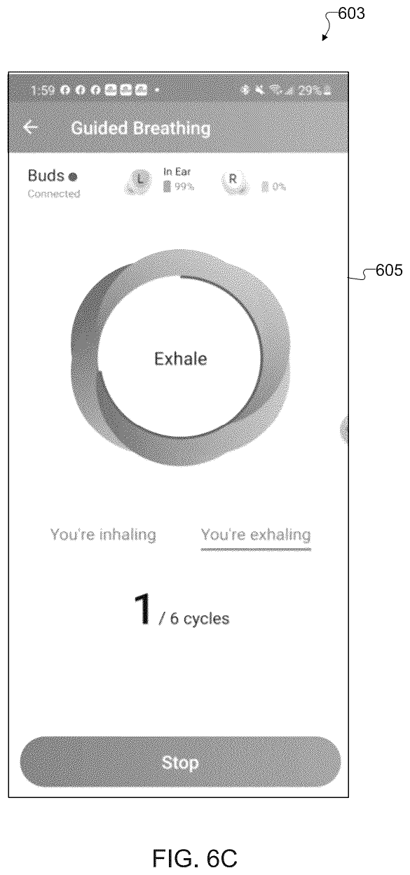

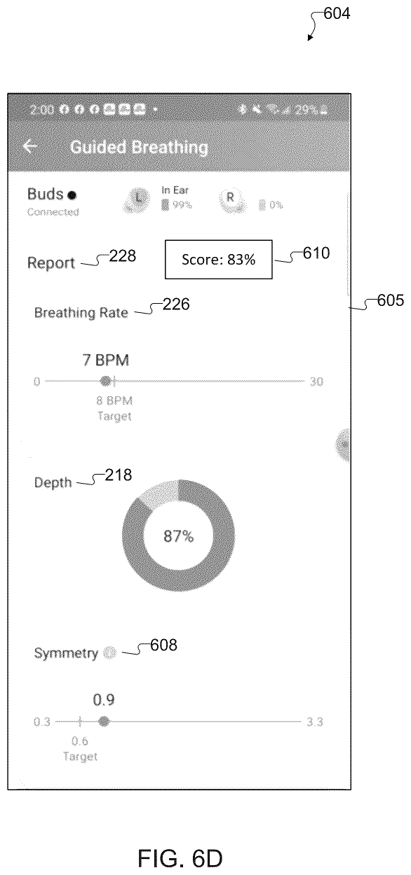

Turning again to FIG. 2, the on-bud module 202 provides information associated with the breathing phase 216 and the breathing depth 218 to the on-phone module 206, such as via wireless transmission. The breathing phase 216 information can include durations of inhale phases, durations of exhale phases, durations of breath holds, and the like. At operation 222, the on-phone module 206 can use the breathing phase 216 information to show the user's current breathing phase 216 on a screen (such as a user interface) of the smartphone 208. For example, FIGS. 6A through 6D illustrate example screen images 601-604 of a user interface 605 that can be used during a guided breathing exercise according to this disclosure.

As shown in FIG. 6A, the user interface 605 allows the user to select a guided breathing exercise by choosing durations for inhalation and exhalation. In FIG. 6B, once the guided breathing exercise is in progress, the user interface 605 guides the user by providing feedback (such as “You are inhaling”) during an inhalation phase. In FIG. 6C, the user interface 605 guides the user by providing other feedback (such as “You are exhaling”) during an exhalation phase. If non-breathing head motion is detected, the user interface 605 can guide the user to adjust the head motion, such as by providing feedback like “head motion detected,” “shallow breathing detected,” and the like. While not specifically shown in FIGS. 6B and 6C, the user interface 605 can also show when the user is in a breath holding phase. Graphical animation (such as an image of a balloon inflating and deflating in synch with the user's breathing) can also be used on the user interface 605 to promote user understanding and guide the user.

At operation 220, the on-phone module 206 uses the timestamps of the breathing phase 216 information to calculate the median inhale and exhale time from the detected phases. For example, the on-phone module 206 may calculate the real-time breathing rate 226 from the median inhalation and exhalation time according to the following.

$$Breathing Rate = \frac{60}{\begin{matrix}median ( inhale duration ) + median ( exhale duration ) + \\ median ( hold duration )\end{matrix}}$$

At operation 224, during the guided breathing process, if the user is not following the guidance, the on-phone module 206 can use the breathing rate 226 information to provide real-time feedback to the user. In the beginning, the user can select a target breathing rate by setting up the intended inhale and exhale duration for the session (such as shown in FIG. 6A). If the estimated real-time breathing rate 226 is greater than a selected threshold (such as two breaths per minute), the on-phone module 206 can instruct the user to follow the guidance on the screen. Using the breathing depth 218 information, the on-phone module 206 can also generate a warning if the user is moving his or her head or not performing deep breathing.

When the user has finished the breathing exercise, the on-phone module 206 can prepare and show a performance report 228 on the screen of the smartphone 208 (such as in the user interface 605). For example. FIG. 6D shows an example of the performance report 228 on the user interface 605. The performance report 228 can show the median breathing rate 226, inhalation to exhalation ratio (breathing symmetry) 608, breathing depth 218, and a breathing performance score 610. The breathing performance score 610 may reflect compliance both in following the animation and in depth of the breathing. The breathing performance score 610 may be weighted to give more credit to a user who can better follow the guidance in order to keep the user engaged in breathing exercises and perform deeper breathing with more compliance. In some embodiments, the on-phone module 206 can compute the breathing performance score 610 using the following.

$$Breathing Performance Score = α * 100 * {e}^{- \frac{1}{3} \vert targetRate - actualRate \vert} + ( 1 - α ) * 1.33 * max ( min ( depth , 100 ) - 24 , 0 )$$

Here, α is a weighting constant. The portion of the formula (α*100*e^{1/3|targetRate-actualRate|}) a calculates the part of the score 610 for the breathing rate 226. If the user maintains the overall target breathing rate throughout the exercise, the user gets full credit, and the point exponentially diminishes if the user does not comply with the guidance. The other portion of the formula ((1−α)*1.33*max(min(depth, 100)−24, 0)) calculates the contributions from the breathing depth 218. This part does not give credit if the breathing depth 218 is very low (such as <25%) and gives full credit if the breathing depth 218 is at or above 100%. The higher the breathing depth 218, the more compliance. The weighting constant α (which may have a value such as 0.65) can be tuned to weight the breathing performance score 610 based on the breathing rate 226 and the breathing depth 218. More credit can be given to the user for meeting the breathing rate target, since it may be more interpretable and clearer to follow from the breathing guidance on the app. Other embodiments can use different thresholds for the breathing depth, different rate factors, or different weights depending on the target use cases and applications.

Although FIGS. 2 through 6D illustrate one example system 200 for tracking and recommending breathing exercises using wearable devices and related details, various changes may be made to FIGS. 2 through 6D. For example, while the processes 300, 350, and 500 shown in FIGS. 3A, 3B, and 5 are described as involving specific sequences of operations, various operations described with respect to FIGS. 3A, 3B, and 5 could overlap, occur in parallel, occur in a different order, or occur any number of times (including zero times). Also, the specific operations shown in FIGS. 3A, 3B, and 5 are examples only, and other techniques could be used to perform each of the operations shown in FIGS. 3A, 3B, and 5. In addition, the user interface 605 shown in FIGS. 6A through 6D could include other types of organized data in other arrangements.

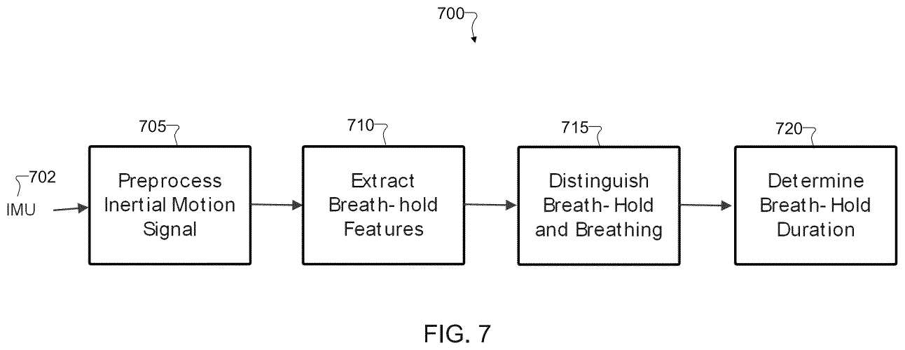

FIG. 7 illustrates an example process 700 for breath-hold detection according to this disclosure. The process 700 can be performed by the on-bud module 202, the on-phone module 206, or a combination of the two during breathing exercises by the user. As shown in FIG. 7, the process 700 obtains motion data 702 from the accelerometer 211a, the gyroscope 211b, or both. At operation 705, the on-bud module 202 performs preprocessing of the motion data 702, such as smoothing each axis of the motion data 702 using a median filter, computing the accelerometer magnitude using a L2-norm algorithm, and applying windowing on both the original axis and the magnitude signal. In some embodiments, a one-second non-overlapping window of motion data 702 may be used. However, other embodiments can use a smaller or larger sliding window. Also, in some embodiments, the preprocessing takes the first differential of the motion data 702 to handle the baseline shift.

At operation 710, the on-bud module 202 extracts multiple breath-hold features related to the breath-hold detection from the motion data 702. Example features can include the percentile range of the motion data 702, overall range of the motion data 702, standard deviation of the motion data 702, and the like. Features can be extracted from each axis and the magnitude signal. The extracted breath-hold features can be used to distinguish the breath-hold from the regular breathing, which can often be shallow in nature. At operation 715, the on-bud module 202 distinguishes between breath-hold and breathing using the extracted features. To classify the breath-hold from breathing, the on-bud module 202 can execute a trained classifier in which the breath-hold features are calculated from regular breathing, deep breathing, and non-breathing head motion as the negative class and breath-holding during breathing exercises as the positive class. The trained classifier can use any suitable machine learning algorithm(s) including (but not limited to) random forest, decision tree, logistic regression, or neural networks. At operation 720, the on-bud module 202 determines the breath-hold duration by clustering the consecutive breath-hold windows together.

Although FIG. 7 illustrates one example of a process 700 for breath-hold detection, various changes may be made to FIG. 7. For example, while shown as a series of steps, various steps in FIG. 7 could overlap, occur in parallel, occur in a different order, or occur any number of times (including zero times). Also, while the process 700 is described above as being performed only by the on-bud module 202, portions of the breath-hold detection process 700 can be performed by the on-phone module 206 as shown in FIG. 2. For instance, operations 705 and 710 can be performed by the on-bud module 202 as part of the breathing phase detection module 212, the extracted features can be provided to the on-phone module 206 as part of the breathing phase 216 information, and the on-phone module 206 can perform operations 715 and 720 as part of the breathing rate estimation operation 220.

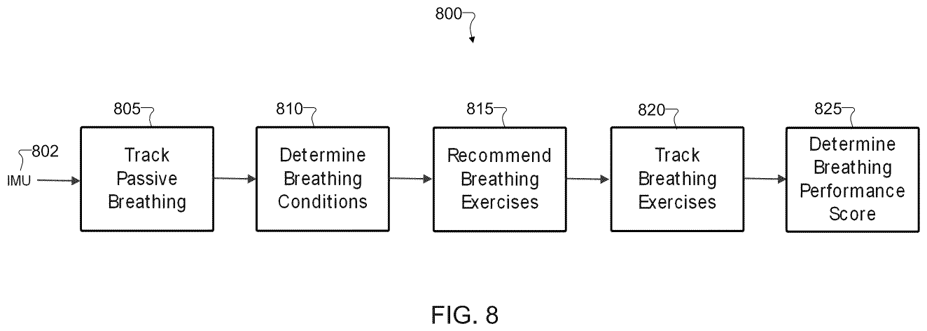

FIG. 8 illustrates an example process 800 for determining passive breathing conditions for breathing exercise recommendation according to this disclosure. The process 800 can be performed to track passive breathing and determine breathing conditions that qualify for breathing exercise recommendation. In addition, the process 800 can track a breathing exercise and provide breathing feedback for a user to self-train on the breathing exercise. For ease of explanation, the process 800 will be described as being performed by the on-phone module 206. However, it will be understood that at least portions of the process 800 can be additionally or alternatively performed by the on-bud module 202.

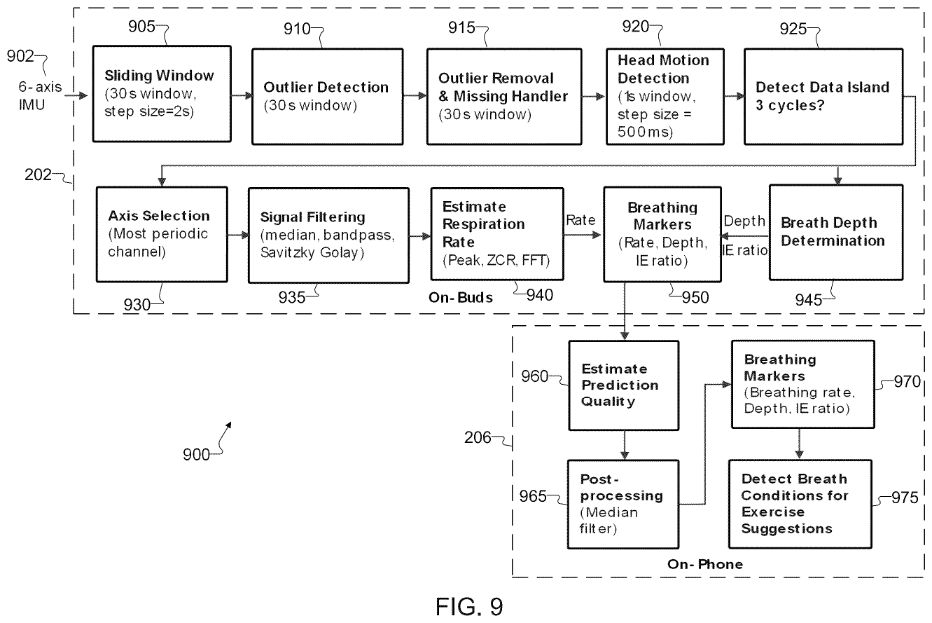

As shown in FIG. 8, at operation 805, the on-phone module 206 tracks passive breathing of the user. This can include times when the user is or is not currently engaged in a breathing exercise. As the user wears one or more earbuds 204, the on-phone module 206 receives motion data 802 from the earbud(s) 204 and extracts one or more passive breathing biomarkers, such as breathing rate, breathing depth, and symmetry (such as inhalation exhalation (IE) ratio). The on-phone module 206 uses the breathing biomarkers to distinguish subtle breathing head motion from heart motion and non-breathing head motion. Further details of passive breathing tracking are described below in conjunction with FIG. 9.

At operation 810, the on-phone module 206 uses the passive breathing biomarkers to determine one or more breathing conditions of the user that qualify for breathing exercise recommendation. Examples of such breathing conditions may include if the user is breathing shallower than expected, if the user is breathing through the mouth for a long period of time, or if the user has a number of breath-hold durations in excess of a threshold during a time period. In some embodiments, the on-phone module 206 can detect shallow breathing by computing the range between the 90^{th} percentile and the 10^{th} percentile of accelerometer X-axis and Z-axis data and comparing the percentile data with pre-trained thresholds. If either of the percentile data are below the thresholds, the on-phone module 206 determines that shallow breathing is detected. In some embodiments, the thresholds can be trained from annotated data collected from different subjects in previous experimentation or studies.

At operation 815, the on-phone module 206 recommends one or more breathing exercises based on the determined breathing conditions. In some embodiments, the on-phone module 206 can select the breathing exercise(s) from a database of breathing exercises that are associated with different breathing conditions. Also, in some embodiments, the on-phone module 206 can determine appropriate customizations for the selected breathing exercise(s) by matching the breathing exercise(s) with the user's baseline breathing rate or the user's breathing score history. For example, if the user's baseline breathing rate is 15 breaths per minute, the on-phone module 206 can recommend that the breathing exercise should start from 10 breaths per minute to lower breathing pace.

At operation 820, the on-phone module 206 tracks the user's breathing exercises as the exercises are being performed, once the exercises are completed, or both. For example, the on-phone module 206 can track the user's breathing exercises using the techniques described in conjunction with FIG. 2. For guided breathing exercise, the on-phone module 206 can provide real-time biofeedback to the user on the detected phase, pace, or symmetry of the on-going breathing exercises. This can be helpful for the user to adjust his or her breathing accordingly to get the best breathing performance score (such as self-training). For meditative exercises, the on-phone module 206 can track the pace of the breathing and provide feedback on the performed breathing rate at the end of meditation. At operation 825, the on-phone module 206 determines the user's breathing performance. For example, for guided breathing exercises, the on-phone module 206 can determine how closely the user followed the target breathing exercises based on the estimated breathing rate, depth, and symmetry. The score can be presented to the user interface as biofeedback and self-training.

Although FIG. 8 illustrates one example of a process 800 for determining passive breathing conditions for breathing exercise recommendation, various changes may be made to FIG. 8. For example, while shown as a series of operations, various operations in FIG. 8 could overlap, occur in parallel, occur in a different order, or occur any number of times (including zero times).

FIG. 9 illustrates an example process 900 for passive breath tracking according to this disclosure. Similar to the process described in FIG. 2, the process 900 includes operations performed by the on-bud module 202 and operations performed by the on-phone module 206. As shown in FIG. 9, the on-bud module 202 uses a sliding window 905 of motion data 902, which may include a three-axis signal from the accelerometer 211a, a three-axis signal from the gyroscope 211b, or a combination of these. In some embodiments, the sliding window 905 is a thirty-second window with a step size of two seconds (thus, consecutive windows 905 overlap by twenty-eight seconds). Other embodiments can include shorter or longer window sizes and overlaps. In general, the size of the sliding window 905 is selected as a trade-off between estimation accuracy and frequency of the breathing rate estimation.

At operation 910, the on-bud module 202 performs outlier detection to detect “outlier” data among the motion data 902. For example, for the sliding window 905, the on-bud module 202 may detect the outliers by calculating the interquartile range (IQR) using the equation IQR=Q3−Q1, where Q1 and Q3 represent the first and third quartiles of the motion data 902 in the sliding window 905. If any value of the motion data 902 is outside of the range [Q1−1.5*IQR, Q3+1.5*IQR], the value will be flagged as outlier. At operation 915, the on-bud module 202 performs outlier removal and missing data handling. For instance, in some cases, the on-bud module 202 may replace an outlier determined in operation 910 with the last non-outlier value or an average value of the data. In other cases, the on-bud module 202 can use winsorization (such as a method of limiting extreme values in statistical data) to handle the outliers. The on-bud module 202 can also use the last non-outlier value to handle any occurrences of missing data.

At operation 920, the on-bud module 202 detects head motion of the user using the motion data 902. In some embodiments, the on-bud module 202 calculates the L2-norm of the three-axis signal from the accelerometer 211a and the three-axis signal from the gyroscope 211b and calculates the range (max−min) or percentile range and standard deviation features on the L2-norms. If any of the feature values is over a certain threshold during a given window of time (such as a one-second window), the on-bud module 202 flags the occurrence as a non-breathing head motion. In some embodiments, the thresholds used in the comparison are trained from annotated data previously collected from multiple subjects in breathing studies or experiments.

At operation 925, the on-bud module 202 performs data island detection to determine if a minimum quantity of data exists in order to perform passive breathing detection. For example, in some algorithms, at least three breathing cycles may be needed for passive breathing biomarker extraction. Here, due to non-breathing head motion and motion artifacts, the on-bud module 202 segments the sliding window 905 into smaller data segments. The on-bud module 202 determines whether at least three or some other number of valid breathing cycles are present in any of the smaller data segments. In some embodiments, the process 900 performs the breathing detection only when this condition is satisfied. This helps to ensure the reliability and quality of the breathing rate and other breathing biomarker estimates.

At operation 930, the on-bud module 202 selects the axis of the motion data 902 with the largest periodic variation, such as determined by Fast Fourier Transformation (FFT), or through zero crossing by the inhalation signal, or by any other suitable technique. At operation 935, the on-bud module 202 performs signal filtering, such as applying a median filter, to smooth the signal. The on-bud module 202 performs z-score normalization on the selected axis and applies band-pass filtering based on the target breathing rate range. The band-pass filter cut-off frequencies can be selected based on the breathing rate of the user. For instance, if the user exhibits a breathing rate in the normal range (such as 10 BPM≤breathing rate≤20 BPM), the on-bud module 202 can apply a second order bandpass filter, such as one with [0.1, 0.5] cut-off frequencies. The on-bud module 202 may apply a third order Savitzky-Golay filter, such as one with a window size of one second, for further smoothing. If the user exhibits a breathing rate in a wider range (such as a breathing rate less than 10 BPM or a breathing rate greater than 20 BPM), the on-bud module 202 can apply a second-order bandpass filter, such as one with [0.02, 0.85] cut-off frequencies. The on-bud module 202 may also apply a third-order Savitzky-Golay filter, such as one with a window size of 0.5 seconds, for further smoothing. In the above, BPM represents breaths per minute.

At operation 940, the on-bud module 202 estimates the user's respiratory rate. In some embodiments, the on-bud module 202 can use one or more of the following algorithms for respiratory rate estimation:
Adaptive ZCR Algorithm (BR_Adaptive): 60/median of zero-crossing duration
Peak Based Algorthm (BR_Peak): 60/median of peak-to-peak distances
FFT Based Algorithm (BR_FFT): 60×maximum amplitude in frequency domain.
   However, these are merely examples, and any other suitable algorithm(s) could be used for estimating the user's respiratory rate.

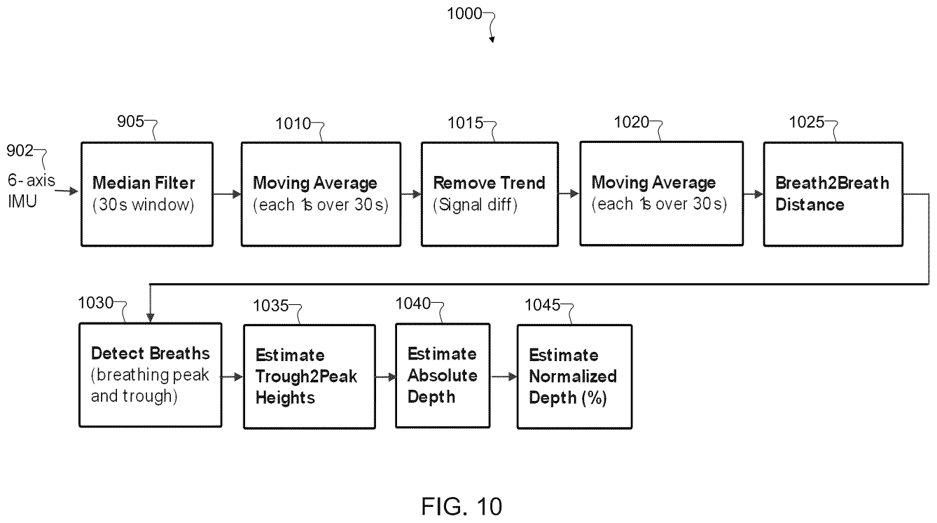

At operation 945, the on-bud module 202 determines the user's breathing depth during passive breathing. In some embodiments, the on-bud module 202 can estimate the breathing depth by just using the accelerometer X-axis data. In other embodiments, the on-bud module 202 can use other axes or combinations of axes. As a particular example, the on-bud module 202 may apply a moving average on the selected axis data and calculates first differentials. The on-bud module 202 may also calculate an average breathing cycle duration, such as by using an FFT or zero-crossing algorithm. This cycle duration is used as a threshold to identify peaks and troughs in the current data island. The breathing depth can be calculated as the amplitude of the inhalation signal, and the breathing depth can be normalized by a factor trained from the training datasets. Further details for breath depth determination are provided below in conjunction with FIG. 10.

During operation 945, the on-bud module 202 can also estimate the inhalation exhalation (IE) ratio. In some embodiments, the on-bud module 202 can use an algorithm that includes the following operations to estimate the IE ratio. Accelerometer Y axis reversal can be performed, such as when the on-bud module 202 reverses the accelerometer Y axis to ensure that inhalation and exhalation at the accelerometer Y axis shows opposite behavior with respect to a chest band (if any) worn by the user during the process 900. Initial peaks and troughs are detected, such as when the on-bud module 202 applies a first-order derivative on the motion data 902, calculates initial peaks and troughs from the positive and negative end points, and calculates a phase amplitude phase_amp and phase duration phase_ts. Certain peaks and troughs are selected, such as when the on-bud module 202 performs phase duration thresholding and adjusts the duration threshold. The on-bud module 202 can calculate an average amplitude factor (λ), such as from the last six amplitudes, and perform amplitude thresholding. Consecutive peaks and troughs can be removed, such as when the on-bud module 202 removes any consecutive peaks with no troughs in between (and vice versa). The IE phase can be calculated, such as when the on-bud module 202 calculates the breathing IE phase from the final peaks and troughs. The IE ratio can also be calculated, such as when the on-bud module 202 calculates the IE ratio from the breathing phase (such as inhalation and exhalation) duration.

At operation 950, the on-bud module 202 collects the various breathing markers (such as the breathing rate, breathing depth. IE ratio, and the like) and transmits the breathing markers to the on-phone module 206 for further processing. At operation 960, the on-phone module 206 estimates the prediction quality of the breathing markers, such as by determining if the standard deviation (BR_Adaptive, BR_Peak. BR_FFT) is less than a selected threshold. At operation 965, the on-phone module 206 performs post-processing on the breathing markers, such as using a median filter to filter signals with heavy head motions and the median of the last three accurate predictions or the like. This results in filtered processed breathing markers 970.

At operation 975, the on-phone module 206 detects one or more breath conditions of the user. Based on the breathing markers 970, the on-phone module 206 can determine if the user exhibits any breath conditions that qualify for breathing exercise recommendation. For example, if a user is having a higher breathing rate compared to his or her baseline for a long period of time, the on-phone module 206 can recommend a breathing exercise. In some embodiments, the on-phone module 206 can input the breathing markers 970 to a trained machine learning model that can determine the breath condition(s), which results in the on-phone module 206 providing the breathing exercise recommendation(s) to the user.

Although FIG. 9 illustrates one example of a process 900 for passive breath tracking, various changes may be made to FIG. 9. For example, while shown as a series of operations, various operations in FIG. 9 could overlap, occur in parallel, occur in a different order, or occur any number of times (including zero times).

FIG. 10 illustrates an example process 1000 for passive breathing depth estimation according to this disclosure. In some embodiments, the process 1000 can be performed as part of the breath depth determination operation 945 of FIG. 9. The process 1000 represents a different algorithm than the process described in FIG. 2, which is associated with real-time guided breathing exercises, because the process 1000 estimates shallower breathing and works on longer window size. In some embodiments, the process 1000 can be performed just using the accelerometer X-axis data. However, other embodiments can use other axes or combinations of axes.

At operation 1010, the on-bud module 202 applies a one-second or other moving average to the sliding window 905 in order to smooth the motion data 902. At operation 1015, the on-bud module 202 takes the first differentials of the motion data 902 in order to remove baseline drift. At operation 1020, the on-bud module 202 again applies a one-second or other moving average to the sliding window 905 in order to further smooth the motion data 902. At operation 1025, the on-bud module 202 estimates breathing cycle duration (such as the temporal distance between breaths). In some embodiments, the on-bud module 202 calculates the breathing cycle duration using FFT and/or zero-crossing algorithms. At operation 1030, the on-bud module 202 uses the cycle duration as a threshold to identify peak and troughs in the current data segment. Part of the trough-to-trough signal is considered one detected breathing cycle.

At operation 1035, the on-bud module 202 estimates trough-to-peak heights for each breath. At operation 1040, the on-bud module 202 estimates absolute breathing depth. Average trough-to-peak height is considered as the absolute breathing depth since this is highly correlated with the tidal volume. At operation 1045, the on-bud module 202 normalizes the breathing depth into a normalized depth, such as by using a factor trained on a training dataset. This factor converts the breathing into a percentage to make the depth value more interpretable for end users. In some embodiments, the expected breathing depth percentage is 100%. A current percentage under 100% indicates shallower breathing, while a current percentage above 100% indicates deeper breathing. However, this interpretation can vary from embodiment to embodiment and use case to use case.

Although FIG. 10 illustrates one example of a process 1000 for passive breathing depth estimation, various changes may be made to FIG. 10. For example, while shown as a series of operations, various operations in FIG. 10 could overlap, occur in parallel, occur in a different order, or occur any number of times (including zero times).

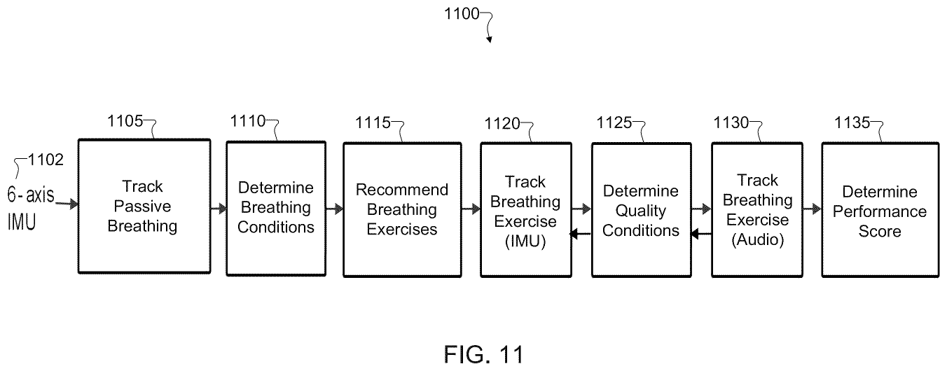

FIG. 11 illustrates an example process 1100 for breath tracking using IMU and audio data according to this disclosure. The embodiments described earlier in this disclosure use low-power inertial sensors (such as the accelerometer 211a and the gyroscope 211b) to track breathing. In contrast, the process 1100 can be performed to track breathing using both IMU data and audio data and determine conditions to switch back and forth between IMU and audio for accuracy and power consumption tradeoff. For ease of explanation, the process 1100 will be described as being performed by the on-phone module 206. However, it will be understood that at least portions of the process 1100 can be additionally or alternatively performed by the on-bud module 202.

As shown in FIG. 11, at operation 1105, the on-phone module 206 tracks the user's passive breathing. This can include times when the user is not currently engaged in a breathing exercise. As the user wears one or more earbuds 204, the on-phone module 206 receives motion data 1102 (IMU data) from the earbud(s) 204 and extracts one or more passive breathing biomarkers (like passive breathing rate, breathing depth, and IE ratio), such as by using the process 900. At operation 1110, the on-phone module 206 uses the passive breathing biomarkers to determine one or more breathing conditions of the user that qualify for breathing exercise recommendation, such as shallow breathing (depth<depth_threshold) or high breathing rate (rate>rate_threshold) for a certain period.

At operation 1115, the on-phone module 206 recommends one or more breathing exercises based on the determined breathing conditions. In some embodiments, the on-phone module 206 recommends the breathing exercise(s) that are appropriate for the baseline breathing pattern, the user's historical breathing performance, and the user's preferences. At operation 1120, if the user starts breathing exercises, the on-phone module 206 tracks the user's breathing (such as rate, phase, depth, IE ratio) in real-time using the low-power motion sensor (IMU).

At operation 1125, the on-phone module 206 determines the quality of the IMU data to track breathing based on non-breathing head motion detection, missing data, signal-to-noise ratio (SNR), and the like. If the quality of the IMU data is not good enough to reliably track the user's breathing, at operation 1130, the on-phone module 206 obtains audio data (such as from an acoustic sensor embedded in the earbuds 204) for breath tracking (which can be performed, for example, using the breath tracking process |200 described below). The process 1100 returns to operation 1125, and the on-phone module 206 determines the quality of the audio data to track breathing. If the environment is too noisy or the breath sound is not audible, the on-phone module 206 switches back to IMU sensing at operation 1120. At operation 1135, at the end of breathing exercise, the on-phone module 206 computes the breathing performance score based on one or more of breathing rate, phase durations, breathing depth, and IE ratio.

As previously noted, breathing exercises can be of two types, namely (i) guided breathing exercises in which the system provides visual, auditory, or audio-visual guidance to the user for a particular exercise suited for the user in the current context and (ii) meditative breathing exercises in which the user performs the breathing exercise on his or her own without any guidance. For guided breathing exercises, the process 1100 can be performed to determine the user's breathing performance score based on a target and the actual breathing. For meditative breathing exercises, the process 1100 can be performed to determine the breathing rate, depth, and IE ratio as part of a report for the user's self-reflection. In some embodiments, a stress score can also be determined and shown to the user before and after the breathing exercises.

Although FIG. 11 illustrates one example of a process 1100 for breath tracking using IMU and audio data, various changes may be made to FIG. 11. For example, while shown as a series of operations, various operations in FIG. 11 could overlap, occur in parallel, occur in a different order, or occur any number of times (including zero times).

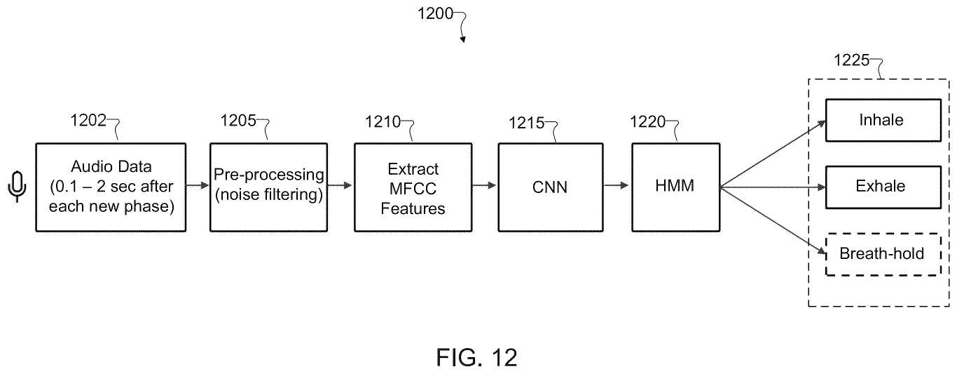

FIG. 12 illustrates an example process 1200 for breath tracking using audio data according to this disclosure. Unlike the process described in FIG. 2 using IMU data to track breathing and the process 1100 described in FIG. 11 using both IMU data and audio data to track breathing, the process 1200 uses only audio data for breath tracking, such as determining breathing phase. For ease of explanation, the process 1200 will be described as being performed by the on-phone module 206. However, it will be understood that at least portions of the process 1200 can be additionally or alternatively performed by the on-bud module 202.

As shown in FIG. 12, at operation 1205, the on-phone module 206 performs pre-processing (such as noise filtering) on audio data 1202 of the user obtained from an acoustic sensor embedded in the earbuds 204. In some embodiments, the audio data 1202 includes between 0.1-2.0 second audio samples. At operation 1210, the on-phone module 206 extracts Mel-frequency cepstral coefficient (MFCC) features from the audio data 1202 and inputs the MFCC features to a convolutional neural network (CNN) 1215. The CNN 1215 can include any suitable architecture, including one or more convolutional layers, max pooling layers, and fully-connected lavers. The CNN 1215 outputs breathing phase information that is input to a hidden Markov model (HMM) 1220, which includes multiple transition probabilities and output probabilities for inhale, exhale, and breath-hold. The HMM 1220 outputs phase information 1225 (such as inhale, exhale, breath-hold, and the like) with probabilities of each phase associated with the audio data 1202.

In some embodiments, the CNN 1215 and the HMM 1220 are trained using multiple breathing sessions from both labs and in-home guided breathing sessions. In some embodiments, the training participants may take a pause in between breath phases. Those pauses can be selected for the “breath-hold” class. In many cases, the training dataset may only need between 0.1-2 second audio samples for training the CNN 1215 for model generalizability and real-time detection. The full breathing sessions can be used to calculate the transition probability of the HMM 1220. To evaluate the performance of the CNN 1215 and the HMM 1220, the training can employ cross-validation (such as ten-fold cross-validation) for model evaluation. The trained models can be evaluated on the rest of the breathing exercises to demonstrate model robustness with different types of breathing exercises.

Although FIG. 12 illustrates one example of a process 1200 for breath tracking using audio data, various changes may be made to FIG. 12. For example, while shown as a series of operations, various operations in FIG. 12 could overlap, occur in parallel, occur in a different order, or occur any number of times (including zero times).

Note that the operations and functions shown in or described with respect to FIGS. 2 through 12 can be implemented in an electronic device 101, 102, 104, server 106, or other device(s) in any suitable manner. For example, in some embodiments, the operations and functions shown in or described with respect to FIGS. 2 through 12 can be implemented or supported using one or more software applications or other software instructions that are executed by the processor 120 of the electronic device 101, 102, 104, server 106, or other device(s). In other embodiments, at least some of the operations and functions shown in or described with respect to FIGS. 2 through 12 can be implemented or supported using dedicated hardware components. In general, the operations and functions shown in or described with respect to FIGS. 2 through 12 can be performed using any suitable hardware or any suitable combination of hardware and software/firmware instructions. Also, the functions shown in or described with respect to FIGS. 2 through 12 can be performed by a single device or by multiple devices.

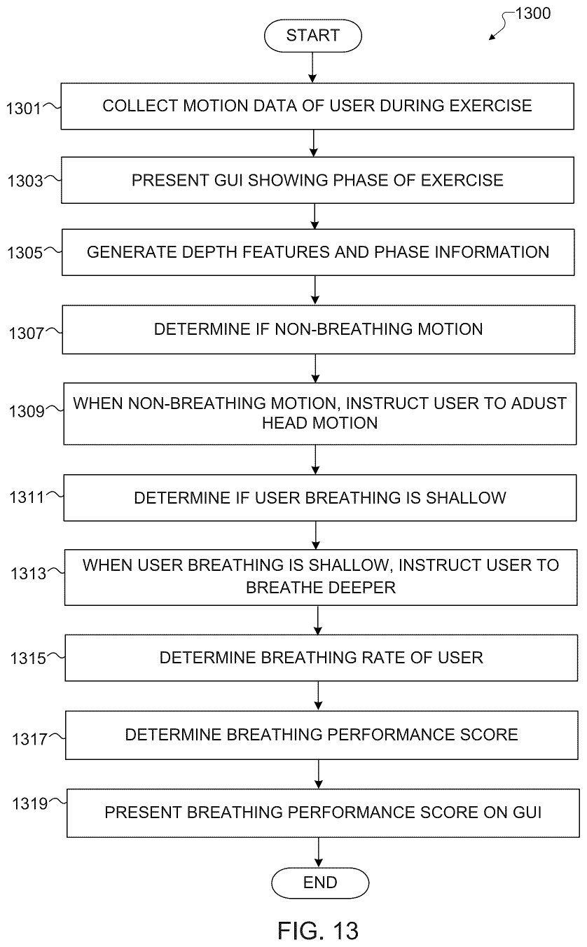

FIG. 13 illustrates an example method 1300 for tracking and recommending breathing exercises using at least one wearable device according to this disclosure. For ease of explanation, the method 1300 shown in FIG. 13 is described as involving the use of the system 200 shown in FIG. 2. However, the method 1300 shown in FIG. 13 could be used with any other system(s).

As shown in FIG. 13, at step 1301, motion data of a user is collected using a head-worn device while the user is performing a breathing exercise. This could include, for example, the motion sensor 210 on one or more earbuds 204 collecting motion data of a user during a breathing exercise. At step 1303, a graphical user interface is presented in real-time as the user is performing the breathing exercise. The graphical user interface shows whether the user is currently in an inhale phase, a breath holding phase, or an exhale phase and a number of breathing cycles completed. This could include, for example, the on-phone module 206 displaying a user interface 605 on the smartphone 208, such as shown in FIGS. 6A through 6D.

At step 1305, for a window of the motion data, breathing depth features and breathing phase information are generated based on the motion data. The breathing phase information indicating durations of inhale phases and durations of exhale phases. This could include, for example, the on-bud module 202 performing the breathing phase detection module 212 and the breathing depth detection module 214 to generate breathing depth features and breathing phase information. At step 1307, using a first machine learning model that receives the breathing depth features as inputs, it is determined whether the motion data corresponds to a non-breathing motion. This could include, for example, the breathing depth detection module 214 performing operation 520 to detect non-breathing head motion. At step 1309, responsive to determining that the motion data corresponds to the non-breathing motion, a first notification is presented to the user to adjust head motion. This could include, for example, the on-phone module 206 presenting a notification on the user interface 605 for the user to adjust head motion.

At step 1311, using a second machine learning model trained to distinguish shallow breathing from deep breathing, it is determined whether the user's breathing is shallow. This could include, for example, the breathing depth detection module 214 performing operation 530 to detect shallow breathing based on the breathing depth features. At step 1313, responsive to determining that the user's breathing is shallow, a second notification is presented to the user to breathe deeper. This could include, for example, the on-phone module 206 presenting a notification on the user interface 605 for the user to breathe deeper.

At step 1315, a breathing rate of the user is determined based on the durations of the inhale phases and the durations of the exhale phases. This could include, for example, the on-phone module 206 determining the user's breathing rate 226. At step 1317, the breathing depth features are used to determine a breathing performance score for the breathing exercise. This could include, for example, the on-phone module 206 determining a breathing performance score 610 for inclusion in a performance report 228. At step 1319, the breathing performance score is presented for the breathing exercise. This could include, for example, the on-phone module 206 presenting the breathing performance score 610 as part of the performance report 228 on the user interface 605.

Although FIG. 13 illustrates one example of a method 1300 for tracking and recommending breathing exercises using a wearable device, various changes may be made to FIG. 13. For example, while shown as a series of steps, various steps in FIG. 13 could overlap, occur in parallel, occur in a different order, or occur any number of times (including zero times).

Although this disclosure has been described with reference to various example embodiments, various changes and modifications may be suggested to one skilled in the art. It is intended that this disclosure encompass such changes and modifications as fall within the scope of the appended claims.

What is claimed is:

1. A method comprising:
collecting motion data of a user using a head-worn device while the user is performing a breathing exercise;
for a window of the motion data, generating breathing depth features based on the motion data;
determining, using a first machine learning model that receives the breathing depth features as inputs, whether the motion data corresponds to a non-breathing motion; and
responsive to determining that the motion data corresponds to the non-breathing motion, presenting a first notification to the user to adjust head motion.

2. The method of claim 1, wherein the motion data is collected using at least one of: a multi-axis accelerometer of the head-worn device and a multi-axis gyroscope of the head-worn device.

3. The method of claim 1, wherein the breathing depth features comprise magnitude and percentile range of the motion data.

4. The method of claim 1, further comprising:
determining whether the user's breathing is shallow by providing the breathing depth features as inputs to a second machine learning model trained to distinguish shallow breathing from deep breathing; and
responsive to determining that the user's breathing is shallow, presenting a second notification to the user to breathe deeper.

5. The method of claim 4, further comprising:
using the breathing depth features from the window of the motion data to determine a breathing performance score for the breathing exercise.

6. The method of claim 1, further comprising:
receiving breathing phase information from the head-worn device while the user is performing the breathing exercise, the breathing phase information indicating durations of inhale phases and durations of exhale phases;
presenting, in real-time as the user is performing the breathing exercise, a graphical user interface showing whether the user is currently in an inhale phase, a breath holding phase, or an exhale phase and a number of breathing cycles completed;
determining a breathing rate of the user based on the durations of the inhale phases and the durations of the exhale phases; and
presenting, on the graphical user interface, a breathing performance score for the breathing exercise based on a comparison of the breathing rate of the user and a target breathing rate for the breathing exercise.

7. The method of claim 6, wherein the breathing exercise comprises inhaling, holding breath, and exhaling during each of the breathing cycles.

8. The method of claim 6, further comprising:
determining a breathing depth of the user based on amplitudes of the motion data; and
comparing the breathing depth of the user to a threshold breathing depth, wherein the breathing performance score is further based on the breathing depth.

9. An electronic device comprising:
at least one processing device configured to:
collect motion data of a user using a head-worn device while the user is performing a breathing exercise;
for a window of the motion data, generate breathing depth features based on the motion data;
determine, using a first machine learning model that receives the breathing depth features as inputs, whether the motion data corresponds to a non-breathing motion; and
responsive to determining that the motion data corresponds to the non-breathing motion, present a first notification to the user to adjust head motion.

10. The electronic device of claim 9, wherein the at least one processing device is configured to collect the motion data using at least one of: a multi-axis accelerometer of the head-worn device and a multi-axis gyroscope of the head-worn device.

11. The electronic device of claim 9, wherein the breathing depth features comprise magnitude and percentile range of the motion data.

12. The electronic device of claim 9, wherein the at least one processing device is further configured to:
determine whether the user's breathing is shallow by providing the breathing depth features as inputs to a second machine learning model trained to distinguish shallow breathing from deep breathing; and
responsive to determining that the user's breathing is shallow, present a second notification to the user to breathe deeper.

13. The electronic device of claim 12, wherein the at least one processing device is further configured to use the breathing depth features from the window of the motion data to determine a breathing performance score for the breathing exercise.

14. The electronic device of claim 9, wherein the at least one processing device is further configured to:
receive breathing phase information from the head-worn device while the user is performing the breathing exercise, the breathing phase information indicating durations of inhale phases and durations of exhale phases;
present, in real-time as the user is performing the breathing exercise, a graphical user interface showing whether the user is currently in an inhale phase, a breath holding phase, or an exhale phase and a number of breathing cycles completed;
determine a breathing rate of the user based on the durations of the inhale phases and the durations of the exhale phases; and
present, on the graphical user interface, a breathing performance score for the breathing exercise based on a comparison of the breathing rate of the user and a target breathing rate for the breathing exercise.

15. The electronic device of claim 14, wherein the at least one processing device is further configured to:
determine a breathing depth of the user based on amplitudes of the motion data; and
compare the breathing depth of the user to a threshold breathing depth, wherein the breathing performance score is further based on the breathing depth.

16. A non-transitory machine-readable medium containing instructions that when executed cause at least one processor of an electronic device to:
collect motion data of a user using a head-worn device while the user is performing a breathing exercise;
for a window of the motion data, generate breathing depth features based on the motion data;
determine, using a first machine learning model that receives the breathing depth features as inputs, whether the motion data corresponds to a non-breathing motion; and
responsive to determining that the motion data corresponds to the non-breathing motion, present a first notification to the user to adjust head motion.

17. The non-transitory machine-readable medium of claim 16, wherein the instructions when executed cause the at least one processor to collect the motion data using at least one of: a multi-axis accelerometer of the head-worn device and a multi-axis gyroscope of the head-worn device.

18. The non-transitory machine-readable medium of claim 17, wherein the instructions when executed further cause the at least one processor to:
determine whether the user's breathing is shallow by providing the breathing depth features as inputs to a second machine learning model trained to distinguish shallow breathing from deep breathing; and
responsive to determining that the user's breathing is shallow, present a second notification to the user to breathe deeper.

19. The non-transitory machine-readable medium of claim 16, further containing instructions that when executed cause the at least one processor to:
receive breathing phase information from the head-worn device while the user is performing the breathing exercise, the breathing phase information indicating durations of inhale phases and durations of exhale phases;
present, in real-time as the user is performing the breathing exercise, a graphical user interface showing whether the user is currently in an inhale phase, a breath holding phase, or an exhale phase and a number of breathing cycles completed;
determine a breathing rate of the user based on the durations of the inhale phases and the durations of the exhale phases; and
present, on the graphical user interface, a breathing performance score for the breathing exercise based on a comparison of the breathing rate of the user and a target breathing rate for the breathing exercise.

20. The non-transitory machine-readable medium of claim 19, further containing instructions that when executed cause the at least one processor to:
determine a breathing depth of the user based on amplitudes of the motion data; and
compare the breathing depth of the user to a threshold breathing depth, wherein the breathing performance score is further based on the breathing depth.
